(19) United States
(12) Patent Application Publication
(10) Pub. No.: US 2009/0233899 A1
(43) Pub. Date: Sep. 17, 2009

(54) Crystalline Modification of Cefuroximaxetil

(75) Inventor: Andreas Fischer, Huertgenwald (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/257,009

(22) Filed: Oct. 23, 2008

Related U.S. Application Data
(63) Continuation of application No. PCT/EP2007/003466, filed on Apr. 20, 2007.

(30) Foreign Application Priority Data
Apr. 25, 2006 (DE) DE102006019619.8

(51) Int. Cl. A61K 31/546; C07D 501/00

(52) U.S. Cl. 514/210.05; 540/222

(57) ABSTRACT

A novel crystalline modification of cefuroximaxetil (ε-modification), pharmaceutical compositions containing this modification, and their use.

### CROSS REFERENCE TO RELATED APPLICATIONS

This application is a continuation of international patent application no. PCT/EP2007/003466, filed Apr. 20, 2007, designating the United States of America, and published in German on Nov. 1, 2007 as WO 2007/121933, the entire disclosure of which is incorporated herein by reference. Priority is claimed based on Federal Republic of Germany patent application no. DE 10 2006 019 619.8, filed Apr. 25, 2006.

### BACKGROUND OF THE INVENTION

The present invention relates to a crystalline modification of cefuroximaxetil (ε-modification), pharmaceutical compositions containing this modification, and their use.

Cefuroximaxetil (ATC code J01DA45) is a broad-spectrum antibiotic of the class of cephalosporins, which is normally administered orally. An administration of cefuroximaxetil is indicated in the case of infections of the respiratory tracts, the genito-urinary tract and in the case of infections in the ear, nose and throat area. Application can also be made in the case of infections in soft tissue and infections in bones and joints (cf. L. J. Scott et al., Drugs 2001, 61, 1455-1500).

Cefuroximaxetil is the 1-acetoxyethylester of cefuroxime, i.e. (6R,7R)-3-carbamoyloxymethyl-7-[(Z)-2-(fur-2-yl)-2-methoxyiminoacetamido]ceph-3-em-4-carboxylic acid (cf. U.S. Pat. No. 3,974,153 and U.S. Pat. No. 4,267,320; ATC code JO1DA06). Where approved for pharmaceutical use, cefuroximaxetil is usually a mixture of two diastereomers. Methods for isolating the individual diastereomers are described in the prior art (cf. DE-OS-27 06 413 and U.S. Pat. No. 5,063,224).

As used herein the term “cefuroximaxetil” preferably refers to the pharmaceutically usable mixture of diastereomers, as defined, for example, in USP 23, pages 315/316. In this case, the two diastereomers are present in a ratio of about 1:1. A sample of the mixture of the diastereomers is chromatographed in accordance with USP 23. A peak r_{A} then corresponds to the A-isomer and a peak r_{B} corresponds to the B-isomer. The ratio of the diastereomers is then determined according to the formula r_{A}/(r_{A}+r_{B}) according to page 315, right column, USP 23. This ratio must lie between 0.48 and 0.55. Various solid modifications of cefuroximaxetil are known in the prior art.

The amorphous form of cefuroximaxetil is usually used to produce oral dosage forms (cf. DE-OS 34 27 828, EP-A 107 276 and U.S. Pat. No. 4,562,181). Various crystalline modifications of cefuroximaxetil are known besides the amorphous form of cefuroximaxetil. Thus, GB-A-15 71 683 discloses a modification, which is referred to hereafter as “α-modification”. An IR spectrum is disclosed in GB-A-21 45 409. EP-A-757 991 discloses a modification, which is referred to hereafter as “β-modification”. EP-A-937 727 discloses a modification, which is referred to hereafter as “γ-modification”. In addition, EP-A-937 727 discloses a hemihydrate, which is composed to about 95% of the R-isomer. DE 10 2005 019 458 describes a modification, which is referred to hereafter as “δ-modification”.

Various polymorphs of the same pharmaceutical active substance differ fundamentally in their properties. On the one hand, the special physical properties of a specific modification can be of interest with respect to its processing and storage such as, for example, thermodynamic stability, crystal morphology [in particular structure, shape, particle size, colour], density, bulk density, hardness, deformability, calorimetric behaviour [in particular melting point], solubility properties [in particular intrinsic solubility rate and equilibrium solubility], hygroscopy, relative humidity profile, stickiness, etc. On the other hand, the crystalline modification can also have improved chemical properties. For example, it is known that a lower hygroscopy can lead to an improved chemical stability and longer durability of chemical compounds.

The hitherto known modifications (polymorphs) of cefuroximaxetil have certain disadvantages. For instance, they do not have an adequate thermodynamic stability, amongst other things. However, the stability of the modification is a most essential aspect in polymorphism. Namely, only by using the most stable modification in the drug can it be assured that no polymorphic transition occurs in the formulation during storage. This is of special importance in that otherwise as a result of a conversion from a less stable modification into a more stable modification the properties of the drug could change. With respect to the pharmacological properties of a dosage form, this means that because of the polymorphic transition, for example, the solubility of the active substance can change, as a result of which a change in the release behavior and thus a change in the bioavailability occurs. Finally, an inadequate storage stability of the dosage form will result from this.

### SUMMARY OF THE INVENTION

It is therefore an object of the invention to provide a crystalline modification of cefuroximaxetil, which has advantages over the modifications of the prior art.

It is also an object of the invention to provide a crystalline modification of cefuroximaxetil which can be produced in a simple manner in larger quantities.

A further object of the invention is to provide a new crystalline modification of cefuroximaxetil which is distinguished in particular by a high thermodynamic stability.

It has been surprisingly found that a novel crystalline modification of cefuroximaxetil can be produced, which has advantages over the crystalline modifications of cefuroximaxetil known hitherto. To distinguish the novel crystalline modification of cefuroximaxetil from the modifications known from the prior art in respect of terminology, the novel crystalline modification is referred to hereafter as “ε-modification”.

It also has been surprisingly found that the ε-modification of cefuroximaxetil according to the invention can be produced, inter alia, by precipitation from a solution of amorphous cefuroximaxetil in acetone by adding water and subsequently stirring the suspension. For this purpose, a filtered solution of amorphous cefuroximaxetil in acetone can be provided and precipitation induced by slowly adding water as precipitant. The amount of water used in this case preferably amounts to onε-times, preferably at least one and a half-times and more preferred at least twice the amount of acetone used. The suspension is then stirred for several hours, the precipitate is filtered out, if necessary, and then dried in a vacuum, if necessary. This method makes it easily possible to also produce the ε-modification of cefuroximaxetil practically in pure form on a gram or kilogram scale.

### BRIEF DESCRIPTION OF THE DRAWINGS

FIG. 1 is an FT Raman spectrum of the modification of cefuroximaxetil (ε-modification) according to the invention; and

FIG. 2 is an X-ray powder diffractogram of the modification of cefuroximaxetil (ε-modification) according to the invention.

### DETAILED DESCRIPTION OF PREFERRED EMBODIMENTS

The crystalline ε-modification of cefuroximaxetil according to the invention is stable in storage, both in the physical and chemical sense.

With the assistance of binary mixtures from the ε-modification with the α-, β- or γ-modification in methanol and acetone it was possible to demonstrate that the ε-modification according to the invention is respectively more stable than the α-, β- or γ-modification under these conditions. Therefore, a spontaneous transformation of the ε-modification into these less stable modifications can be excluded on thermodynamic grounds.

As a result of the fact that further crystalline modifications are also already known for cefuroximaxetil besides the amorphous modification and the crystalline ε-modification according to the invention, this aspect is additionally significant. Namely, cefuroximaxetil is evidently distinguished by a pronounced tendency towards polymorphism, which is not the case with every drug. As a result of the comparatively large number of metastable modifications, however, a polymorphic transition during storage is fundamentally more likely. In such cases, therefore, there is a particular requirement to provide the most stabile polymorphic modification where possible.

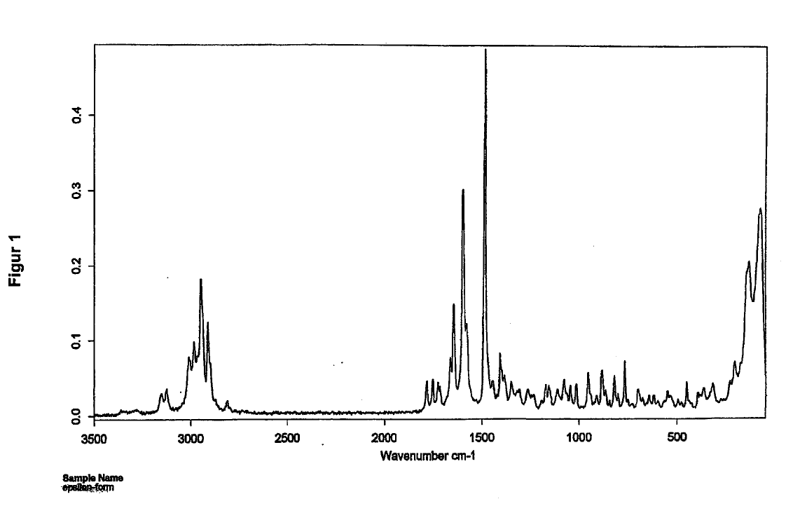

An FT Raman spectrum of the novel crystalline modification of cefuroximaxetil (ε-modification) is shown in FIG. 1. The FT Raman spectrum of the crystalline modification of cefuroximaxetil (ε-modification) according to the invention comprises signals at 1661±3 (preferably ±2, more preferred ±1) cm⁻¹, 1644±3 (preferably ±2, more preferred ±1) cm⁻¹ and 1597±3 (preferably ±2, more preferred ±1) cm⁻¹. The FT Raman spectrum preferably additionally comprises a signal at 1404±3 (preferably ±2, more preferred ±1) cm⁻¹ and/or 1349±3 (preferably ±2, more preferred ±1) cm⁻¹.

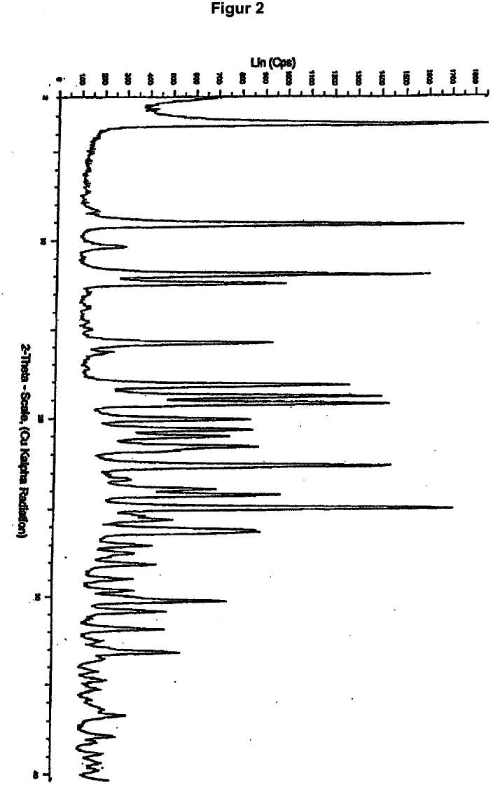

An X-ray powder diffractogram of the novel crystalline modification of cefuroximaxetil (ε-modification) is shown in FIG. 2. The X-ray powder diffractogram of the ε-modification according to the invention preferably comprises reflexes at 23.35±0.20 (preferably ±0.15, more preferred ±0.10) °2Θ and/or 33.56±0.20 (preferably ±0.15, more preferred ±0.10) °2Θ. The X-ray powder diffractogram preferably additionally comprises reflexes at 15.75±0.20 (preferably ±0.15, more preferred ±0.10) °2Θ, 18.76±0.20 (preferably ±0.15, more preferred ±0.10) °2Θ, 24.18±0.20 (preferably ±0.15, more preferred ±0.10) 020, 24.92±0.20 (preferably ±0.15, more preferred ±0.10) °2Θ and/or 26.18±0.20 (preferably ±0.15, more preferred ±0.10) °2Θ.

A further aspect of the invention relates to a method for the production of a crystalline modification of cefuroximaxetil comprising the steps
a) dissolving amorphous cefuroximaxetil in a sufficient volume of acetone;
b) adding water to the solution obtained in step a) in a quantity sufficient to precipitate the cefuroximaxetil;
c) stirring the suspension formed;
d) if necessary, filtering off the precipitate formed; and
e) if necessary, drying the precipitate formed.

The crystalline modification of cefuroximaxetil is preferably the abovε-described ε-modification. In the method according to the invention, the amorphous form of cefuroximaxetil is used as educt.

The solution obtained in step a) is preferably filtered before water is added as precipitant in step b). The solution obtained in step a) preferably has a specific concentration of the amorphous form of cefuroximaxetil in acetone in the range of 5.0 to 15% by wt., more preferred 7.5 to 12.5% by wt. and particularly preferred 9.0 to 11% by wt.

In step b) of the method according to the invention, water is added to the solution obtained in step a) in a quantity sufficient to precipitate the cefuroximaxetil. The addition of water as precipitant is preferably performed slowly, preferably in drops over a longer period of time so that the precipitation is not caused abruptly but carefully. The addition preferably occurs at room temperature. Depending on the quantities used, a person skilled in the art knows what period of time is appropriate for the addition of water, in particular so that occlusions and inclusions are also prevented.

The added quantity of water is sufficient to induce precipitation of the cefuroximaxetil. The ratio of the volume of water to the volume of acetone preferably lies in the range of 1:1 to 5:1, more preferred 1.5:1 to 4:1, further preferred 1.75:1 to 3:1.

In step c) of the method according to the invention the suspension obtained in step b) is stirred. The stirring is preferably performed for at least 6 h, more preferred for at least 12 h, further preferred at least 18 h, and in particular at least 24 h, preferably at room temperature.

In the optional step e) of the method according to the invention, a drying operation is conducted, preferably in a vacuum and/or at elevated temperature, preferably in the range from 30° C. to 50° C., more preferred 35° C. to 45° C. and in particular at 40° C.

In an alternative embodiment of the method according to the invention the α-modification of cefuroximaxetil is worked from as educt and not the amorphous form of cefuroximaxetil. However, in this case it is not a solution in acetone, but a solution in THF that is prepared and precipitation is initiated by adding water as precipitant. The amount of water added is sufficient to induce the precipitation of the cefuroximaxetil. The ratio of the volume of water to the volume of THF is preferably in the range of 0.5:1 to 2:1, more preferred 0.75:1 to 1.5:1, further preferred 0.8:1 to 1:1.

Under these conditions an oily precipitate can firstly form, which, however, transforms into the crystalline ε-modification upon storage for several days, preferably a week, at room temperature. This can then be filtered out and dried under a vacuum, if necessary.

A further aspect of the invention relates to the crystalline modification of cefuroximaxetil, which can be obtained by means of one of the abovε-described methods.

A further aspect of the invention relates to pharmaceutical compositions, which comprise the modification of cefuroximaxetil (ε-modification) according to the invention and a pharmaceutically acceptable support.

In one preferred embodiment, the composition according to the invention contains at least one carrageenan, which is preferably selected from the group comprising κ-carrageenan, λ-carrageenan and ι-carrageenan, preferably κ-carrageenan. Carrageenans are sulfatised polysaccharides, which occur in the form of structural components in the cell walls of certain red algae species. κ-carrageenan can be obtained, for example, from Kappaphycus alverezii, Chondrus crispus and Sarcothalia crispata.

κ-carrageenan, λ-carrageenan and ι-carrageenan are polysaccharides, which differ in particular in the number and position of sulfate ester groups. Unless otherwise specified, the term “carrageenan” in the sense of the description preferably means at least a carrageen selected from the group comprising κ-carrageenan, λ-carrageenan, and ι-carrageenan. However, there are even more carrageenans known from the prior art, e.g. α-carrageenan, β-carrageenan, γ-carrageenan and ε-carrageenan.

A lead structure of κ-carrageenan can preferably also be expressed chemically as “-(1→3)-β-D-galactopyranosε-4-sulfatε-(1→4)-3,6-anhydro-α-D-galactopyranosε-(1→3)-”, the lead structure of ι-carrageenan as “-(1→3)-β-D-galactopyranosε-4-sulfatε-(1→4)-3,6-anhydro-α-D-galactopyranosε-2-sulfatε-(1→3)-” and the lead structure of λ-carrageenan as “-(1→3)-β-D-galactopyranosε-2-sulfatε-(1→4)-α-D-galactopyranosε-2,6-disulfatε-(113)-”.

In accordance with the present invention, κ-carrageenan preferably comprises sulfatised polygalactoside substructures, which are derived from D-galactose and 3,6-anhydro-D-galactose. Examples of such substructures include:

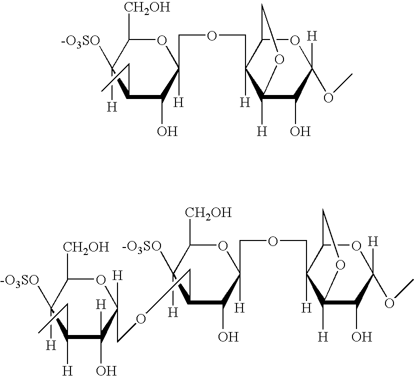

The composition according to the invention preferably contains a carrageenan, which has less than 1.5 sulfate equivalents, preferably less than 1.0 sulfate equivalent and in particular 0.6±0.1 sulfate equivalents on average per pyranose unit.

The composition according to the invention preferably contains κ-carrageenan, preferably as sodium, potassium or calcium salt. κ-carrageenan and its salts are commercially available, e.g. under the name Gelcarin®. For further details concerning κ-carrageenan and for the specification of carrageenans, reference can be made, for example, to T.H.M. Snoeren, Kappa-carrageenan: A study on its physico-chemical properties, sol-gel transition and interaction with milk proteins (N.I.Z.O.-verslagen), H. Veenman and Zonen B. V (1976); Uno Y, Omoto T, Goto Y, Asai I, Nakamura M, Maitani T (2001), Molecular weight distribution of carrageenans studies by a combined gel permeation/inductively coupled plasma (GPC/ICP) method, Food Additives and Contaminants 18: 763-772; and JECFA (1998), Compendium of Food Additive Specifications Addendum 6, pp 29-33. FAO, Rome.

In a preferred embodiment of the composition according to the invention, it contains at least 5.5% by wt., preferably at least 6.0% by wt., further preferred at least 6.5% by wt., most preferred at least 7.0% by wt., and in particular at least 7.5% by wt. carrageenan, preferably κ-carrageenan. It is particularly preferred if the composition according to the invention contains 7.5 to 50% by wt., preferably 10 to 40% by wt. and in particular 20 to 30% by wt. carrageenan, preferably κ-carrageenan, calculated on the basis of the total weight of the composition.

The weight ratio of the ε-modification of cefuroximaxetil to carrageenan preferably lies in the range of 0.5:1 to 5.0:1, more preferred 1.0:1 to 4.0:1, further preferred 1.5:1 to 3.5:1, most preferred 2.0:1 to 3.0:1, and in particular 2.2:1 to 2.6:1.

In a preferred embodiment, the composition according to the invention contains tricalcium phosphate and/or at least one sucrose ester. In a particularly preferred embodiment, the composition according to the invention contains both tricalcium phosphate and at least one sucrose ester. Preferred sucrose esters have a hydrophilic-lipophilic balance (HLB) value of 10 to 17, more preferred 11 to 16, in particular 13 to 15. The composition according to the invention preferably contains sucrose ester in a quantity of 1.0 to 10% by wt., more preferred 2.0 to 8.0% by wt. and in particular 4.0 to 6.0% by wt., calculated on the basis of the total weight of the composition.

Examples of sucrose esters in accordance with the present invention include sucrose stearate, sucrose palmitate, sucrose laurate, sucrose behenate, sucrose oleate, sucrose erucate or mixtures thereof. It is particularly preferred if the sucrose ester comprises sucrose palmitate, which has an HLB of approx. 15 and is commercially available, for example, under the designation “S-1570” from Mitsubishi.

If the composition according to the invention contains tricalcium phosphate and carrageenan, then the weight ratio of tricalcium phosphate to carrageenan preferably amounts to 1:1 to 1:10, in particular 1:2 to 1:6. A finely dispersed powder with an average particle size of <50 μm is preferably used as tricalcium phosphate.

In addition, the composition according to the invention can contain usual auxiliary substances such as e.g. fillers, binders, slip agents, antioxidants, colouring agents or preservatives. Such auxiliary substances are known to the person skilled in the art. In this context, for example, reference can be made in full to H. P. Fiedler, Lexikon der Hilfsstoffe für Pharmazie, Kosmetik und angrenzende Gebiete, 2 Bde., Editio Cantor, Aulendorff, 2002; D. E. Bugay, Pharmaceutical Excipients (Drugs and the Pharmaceutical Sciences, V 94) (Hard cover), CRC, 1998.

In one preferred embodiment, the composition according to the invention contains no microcrystalline cellulose or other spheronisers such as e.g. low-substituted hydroxypropyl cellulose, hydroxypropylmethyl cellulose, hydroxymethyl cellulose, powder cellulose, sodium carboxymethyl cellulose or polyvinyl pyrrolidone, where as dosage form they have not yet been provided with a coating.

A further aspect of the invention relates to pellets, preferably extruded pellets, which contain the composition according to the invention.

Yet another aspect of the invention relates to a solid pharmaceutical dosage form containing the ε-modification of cefuroximaxetil according to the invention, wherein the cefuroximaxetil is released under physiological conditions at a pH value of 6 to 7 within 30 minutes in a quantity of at least 75%, preferably 85%, after any coating present has firstly been dissolved. The dosage form is preferably multiparticulate.

In addition, a further aspect of the invention relates to a preferably multiparticulate pharmaceutical dosage form comprising an abovε-described pharmaceutical composition, preferably abovε-described pellets.

Preferred embodiments of the dosage form according to the invention, such as e.g. preferred quantity ratios of the components used, are described above in association with the pharmaceutical composition according to the invention.

The dosage form according to the invention is preferably manufactured for oral administration. It is preferably multiparticulate. It preferably comprises granulates, pellets, preferably extruded pellets, micro-granulates or micropellets. The dosage form according to the invention is preferably provided in the form of extruded pellets, preferably in spherical form. These can be filled into gelatine capsules, if required.

The particles of the multiparticulate dosage form according to the invention preferably have an average diameter (size) of <1000 μm, more preferred <900 μm, further preferred <800 μm, most preferred <700 μm and in particular between 250 and 700 μm.

In a preferred embodiment the dosage form according to the invention is provided in tablet form, in particular as a film tablet. It is particularly preferred in this case if the tablets can be obtained by tabletting the pellets according to the invention, preferably extruded pellets, by adding the usual auxiliary substances for tabletting.

In one preferred embodiment the dosage form according to the invention has a gastric juicε-resistant and/or saliva-resistant coating. Before application of this coating, a protective coating that isolates the core is preferably additionally applied. Depending on the type and function of the coating, these coatings are preferably applied in a quantity of 1.0 to 50% by wt., more preferred 2.0 to 2.5% by wt., calculated on the basis of the total weight of the dosage form. Examples of suitable materials for a gastric juicε-resistant coating include methacrylic acid/alkylmethacrylate copolymers, preferably copolymerisates of methacrylic acid/methylmethacrylate with a molar ratio of the monomers of 1:1 to 1:2, such as Eudragit L®, Eudragit S® or Eudragit L30D-55®, or ethylmethacrylate, which dissolve quickly with a pH value of ≧pH 6. In addition, cellulosε-based coatings that are known to the person skilled in the art can be applied as gastric juicε-resistant coating. Coatings can be applied with corresponding solutions or dispersions in organic or aqueous medium, wherein an aqueous medium is preferred. Suitable saliva-resistant coatings are coatings based on Eudragit E, Eudragit EPO.

It is known to the person skilled in the art that usual softeners, coloring agents, slip agents such as talcum and/or magnesium stearate should and can be added.

As used herein, the term “gastric juice” should be understood to mean both the natural composition of the gastric juice and the synthetic preparations (pH 1.2 to 2.0) similar to gastric juice known to the specialist. Similarly, the term “small intestine release” is intended to refer to both release in the natural small intestine juice and release in preparations similar to small intestine juice at pH values of 6 to 7, preferably pH 6.4 to 6.8.

The dosage forms according to the invention are preferably distinguished in that they have a high dissolution rate, and the cefuroximaxetil is preferably released to at least 85% within 30 minutes after any coating present has firstly dissolved. The pH-dependent dissolution period of such a coating can be determined by simple preliminary tests in corresponding standard buffer solutions.

The release rate of cefuroximaxetil is preferably determined using the method described in “Guidance for Industry, Waiver of In Vivo Bioavailability and Bioequivalence Studies for Immediate-Release Solid Oral Dosage Forms based on a Biopharmaceutics Classification System., pages 1-3/7, editor U.S. Department of Health and Human Services, Food and Drug Administration, Center for Drug Evaluation and Research (CDER), August 2000, BP”. For this, a release apparatus with paddle agitator according to U.S. pharmacopoeia is used and the release is measured at a temperature of 37° C. and a rotational speed of 150 min⁻¹ in 1000 ml of synthetic intestine juice (sodium phosphate buffer, pH 6.8) and 0.3% SDS or in 1000 ml of synthetic gastric juice (pH 1.2) and 0.5% Tween 80 as release medium. The quantity of active substance respectively released at one time can be determined by HPLC. The detection can be achieved using a UV detector at 282 nm.

The active substance content of the dosage form according to the invention is preferably adapted to a once or twice daily administration for an adult or a pediatric patient. In the case of adults, the daily dose is usually in the range of 250 to 1000 mg per day, in children usually in the range of 80 to 500 mg per day, depending on age and weight. The dosage form according to the invention preferably comprises an equivalent quantity per dose unit in relation to 125, 250 or 500 mg of cefuroxime.

In one preferred embodiment, after any coating present has firstly dissolved, the cefuroximaxetil dosage form according to the invention is released at a pH value of 6 to 7 within 30 minutes in a quantity of at least 75%, preferably at least 80%, more preferred at least 85%, and most preferred at least 90%, and in particular at least 95%, determined using the method described in “Guidance for Industry, Waiver of In Vivo Bioavailability and Bioequivalence Studies for Immediate-Release Solid Oral Dosage Forms based on a Biopharmaceutics Classification System., pages 1-3/7, editor U.S. Department of Health and Human Services, Food and Drug Administration, Center for Drug Evaluation and Research (CDER), August 2000, BP”.

In a particularly preferred dosage form according to the invention, the multiparticulate dosage form is provided in the form of spherical extruded pellets. These can preferably be provided as single dose using a delivery system comprising a drinking straw with a preferably movable blocking device such as that described in WO 03/079957, WO 2005/000202, WO2004/000264. The blocking device is preferably a plug with a cross-section preferably adapted to the cross-section of the drinking straw. The plug can be movable between two stops. The drinking straw is narrowed because of the stops, wherein the narrowed sections are constructed so that the plug is thus held back, but not the multiparticulate dosage form. Administration to the patient preferably occurs with a transport fluid. The descriptions of the dosage systems in the specified publications are incorporated herewith by reference and apply as part of the present disclosure.

The present invention further relates to a delivery system comprising a dosage form according to the invention, preferably as single dose arranged in a drinking straw with at least one preferably movable blocking device for administration by means of a transport fluid to a human patient.

Preferred transport fluids are particlε-free beverages, especially aqueous liquids such as e.g. water, tea, fruit juices, lemonades, wherein transport fluids with an acid pH value are particularly preferred when dosage forms equipped to be gastric juicε-resistant, i.e. coated, multiparticulate dosage forms such as e.g. extruded pellets, are used.

The crystalline modification of cefuroximaxetil according to the invention is suitable as an active pharmaceutical substance, preferably for the production of a pharmaceutical composition for the treatment and/or inhibition of infection, i.e. infections caused by cefuroximε-sensitive pathogens, in particular infections of the upper respiratory tracts including the throat and ear region (e.g. otitis media, sinusitis, pharyngitis, tonsilitis), infections of the lower respiratory tracts (e.g. acute exacerbation of chronic bronchitis, pneumonia), infection of the ENT region, bones, joints and in the case of septiceamia, infections of the skin and soft tissue, infections of the kidneys and/or the urinary tracts, sexual organs, acute uncomplicated gonorrhoea, or for perioperative prophylaxis when patients are at increased risk through infections.

The following examples serve to illustrate the invention in further detail, but are not to be interpreted as restrictive to the scope of the invention:

### Example 1

### Production of the Crystalline Modification of Cefuroximaxetil According to the Invention

a) 505.1 mg of amorphous cefuroximaxetil were dissolved in 6 ml of acetone. 9 ml of water were added in stages. The suspension formed was stirred overnight. The precipitate was filtered off and dried in a vacuum at 40° C. It was possible to prove by FT Raman spectroscopy that it was the ε-modification according to the invention.

b) 501.2 mg of amorphous cefuroximaxetil were dissolved in 6 ml of acetone. The solution was filtered and 9 ml of water were added in stages. The suspension formed was stirred overnight. The precipitate was filtered off and dried in a vacuum at 40° C. It was possible to prove by FT Raman spectroscopy that it was the ε-modification according to the invention.

c) 3 g of amorphous cefuroximaxetil were dissolved in 25 ml of acetone. The solution was filtered and 30 ml of water were added in stages. The suspension formed was stirred overnight. The precipitate was filtered off and dried in a vacuum at 40° C. It was possible to prove by FT Raman spectroscopy that it was the ε-modification according to the invention.

d) 515.9 mg of cefuroximaxetil of the α-modification were dissolved in 3 ml of THF. 2.5 ml of water were added in stages. An oily precipitate was obtained, which after storage for a week at room temperature transformed into a solid. The solid was filtered off and dried in a vacuum. It was possible to prove by FT Raman spectroscopy that it was the ε-modification according to the invention.

### Example 2

The crystalline modification of cefuroximaxetil (ε-modification) according to the invention obtained in Example 1a) was identified by FT Raman spectroscopy. For this an FT Raman spectrum, shown in FIG. 1, was imaged on an FT raman spectrometer from Bruker, type RFS100 (Nd:YAG 1064 nm stimultion, 100 mW laser, Gε-detector, 64 scans, 25-3500 cm⁻¹, resolution 2 cm⁻¹).

The measured Raman signals (λ [cm⁻¹]) of the ε-modification according to the invention are recorded in the following table and compared to the corresponding signals of the α-, β- and γ-modification:

| ε | α | β | γ | E | α | β | γ |
|---|---|---|---|---|---|---|---|
| 2949 | | | | 1485 | | | |
| | | 2948 | | | 1484 | | |
| | 2946 | | | | | | 1483 |
| | | | 2944 | | | 1482 | |
| | | 1786 | | | 1405 | | 1405 |
| 1780 | 1780 | | | 1404 | | | |
| | | | 1778 | | | 1398 | |
| | | 1676 | | | 1394 | | 1394 |
| 1661 | | | | 1349 | | | |
| | | 1655 | | | 1082 | | 1082 |
| | | | 1652 | | | 1079 | |
| | 1646 | | | 1076 | | | |
| 1644 | | | | 953 | 953 | | |
| | 1634 | | 1634 | | | 887 | 887 |
| | | 1631 | | | | 771 | |
| | 1599 | | | | | | 770 |
| 1597 | | | | | 769 | | |
| | | 1592 | | 768 | | | |
| | 1584 | | 1584 | | | | |

### Example 3

The crystalline modification of cefuroximaxetil (ε-modification) according to the invention was identified by X-ray diffractometer. For this, an X-ray powder diffractogram (XRPD) with a diffractometer from Philips, type X'Pert PW 3040 (or Philips PW 1710) (Cu K_{α} radiation, ambient temperature and ambient humidity, step size 0.02 °2Θ, 2 or 2.4 s step⁻¹, 2-50 °2Θ) was recorded, which is shown in FIG. 2.

The measured reflexes (°2Θ values) of the ε-modification according to the invention are recorded in the following table:

| °2 Θ | rel. int. |
|---|---|
| 3.40 | 90 |
| 8.31 | 10 |
| 9.07 | 95 |
| 10.31 | 17 |
| 11.89 | 92 |
| 12.37 | 56 |
| 13.74 | 8 |
| 15.75 | 54 |
| 16.28 | 15 |
| 18.14 | 72 |
| 18.76 | 82 |
| 19.16 | 83 |
| 20.02 | 48 |
| 20.60 | 49 |
| 20.96 | 44 |
| 21.54 | 50 |
| 21.74 | 30 |
| 22.57 | 87 |
| 23.35 | 19 |
| 23.84 | 41 |
| 24.18 | 60 |
| 24.92 | 100 |
| 25.36 | 23 |
| 25.57 | 29 |
| 26.18 | 51 |
| 27.01 | 25 |
| 27.45 | 20 |
| 28.08 | 27 |
| 28.93 | 21 |
| 29.62 | 21 |
| 30.21 | 44 |
| 30.82 | 28 |
| 31.82 | 29 |
| 32.52 | 14 |
| 33.13 | 32 |
| 33.56 | 13 |
| 34.26 | 11 |
| 34.77 | 14 |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A crystalline modification of cefuroximaxetil having a FT Raman spectrum which comprises signals at 1661±3 cm⁻¹, 1644±3 cm⁻¹ and 1597±3 cm⁻¹.

2. A crystalline modification of cefuroximaxetil as claimed in claim 1, wherein the FT Raman spectrum additionally comprises a signal at 1404±3 cm⁻¹, or a signal at 1349±3 cm⁻¹, or both.

3. A crystalline modification of cefuroximaxetil as claimed in claim 1, having an X-ray powder diffractogram exhibiting reflections at 23.35±0.20°2Θ, or 33.56±0.20°2Θ, or both.

4. A crystalline modification of cefuroximaxetil as claimed in claim 3, wherein the X-ray powder diffractogram further exhibits at least one reflection selected from the group consisting of 15.75±0.20°2Θ, 18.76±0.20°2Θ, 24.18±0.20°2Θ, 24.92±0.20°2Θ and 26.18±0.20°2Θ.

5. A method of producing a crystalline modification of cefuroximaxetil, said method comprising:
a) dissolving amorphous cefuroximaxetil in acetone to obtain an acetone solution;
b) adding water to the solution obtained in a) in a quantity sufficient to precipitate the cefuroximaxetil and form a suspension;
c) stirring the suspension obtained in b);
d) optionally filtering out the precipitated cefuroximaxetil; and
e) optionally drying the filtered cefuroximaxetil.

6. A method as claimed in claim 5, wherein the produced crystalline modification of cefuroximaxetil has an FT Raman spectrum which comprises signals at 1661±3 cm⁻¹, 1644±3 cm⁻¹ and 1597±3 cm⁻¹.

7. A method as claimed in claim 6, wherein the FT Raman spectrum additionally comprises a signal at 1404±3 cm⁻¹, or a signal at 1349±3 cm⁻¹, or both.

8. A method as claimed in claim 5, wherein the produced crystalline modification of cefuroximaxetil has an X-ray powder diffractogram exhibiting reflections at 23.35±0.20°2Θ, or 33.56±0.20°2Θ, or both.

9. A method as claimed in claim 8, wherein the X-ray powder diffractogram further exhibits at least one reflection selected from the group consisting of 15.75±0.20°2Θ, 18.76±0.20°2Θ, 24.18±0.20°2Θ, 24.92±0.20°2Θ and 26.18±0.20°2Θ.

10. A method as claimed in claim 5, wherein in b) the ratio of the volume of water to the volume of acetone lies in the range of 1:1 to 5:1.

11. A method as claimed in claim 5, wherein the stirring in c) is carried out for at least 6 hours.

12. A method as claimed in claim 5, wherein the drying in e) occurs under a vacuum or at an elevated temperature, or both.

13. A method as claimed in claim 12, wherein the drying in e) is effected at a temperature in the range from 35° C. to 45° C.

14. A crystalline modification of cefuroximaxetil obtained by the method of claim 5.

15. A pharmaceutical composition comprising a crystalline modifications of cefuroximaxetil as claimed in claim 1, and at least one pharmaceutically acceptable carrier or auxiliary substance.

16. A pharmaceutical dosage form comprising a pharmaceutical composition according to claim 15, in an amount sufficient to provide from 80 to 1000 mg of cefuroximaxetil, wherein said dosage form is orally administrable.

17. A method of treating or inhibiting a bacterial infection in a subject, said method comprising administering to said subject an effective antibacterial amount of a crystalline modification of cefuroximaxetil as claimed in claim 1.
